(19) United States
(12) Patent Application Publication
(10) Pub. No.: US 2003/0143560 A1
(43) Pub. Date: Jul. 31, 2003

(54) Mutant DNA encoding peroxisome proliferator-activated receptor-gamma coactivator-1

(76) Inventors: Gitte Andersen, Frederiksberg C (DK); Jakob Ek, Kobenhavn O (DK); Torben Hansen, Hellerup (DK); Oluf Borbye Pedersen, Lyngby (DK)

Correspondence Address: Reza Green, Esq., Novo Nordisk of North America, Inc., 405 Lexington Avenue, Suite 6400, New York, NY, 10174-6401, US

(21) Appl. No.: 10/166,412

(22) Filed: Jun. 7, 2002

Related U.S. Application Data
(60) Provisional application No. 60/296,920, filed on Jun. 8, 2001.
(60) Provisional application No. 60/304,378, filed on Jul. 10, 2001.

(30) Foreign Application Priority Data
Jul. 10, 2001 (DK) PA 2001 01080
Jun. 8, 2001 (EP) EP 01610061.2

(51) Int. Cl. A01K 67/00; C12Q 1/68; C07H 21/04; C12P 21/02; C12N 5/06; C07K 14/705

(52) U.S. Cl. 435/6; 800/8; 435/69.1; 435/320.1; 435/325; 530/350; 536/23.2

(57) ABSTRACT

The present invention relates to a mutant DNA sequence encoding peroxisome proliferator-activated receptor-γ coactivator-1 (PGC-1), a method of detecting a mutation in the gene encoding peroxisome proliferator-activated receptor-γ coactivator-1, as well as a diagnostic composition and a test kit for use in the method.

### CROSS REFERENCE TO RELATED APPLICATIONS

This application claims priority under 35 U.S.C. 119 of Danish application no. PA 2001 01080 filed on Jul. 10, 2001, U.S. provisional application Ser. No. 60/296,920 filed on Jun. 8, 2001, U.S. provisional application no. 60/304,378 filed on Jul. 10, 2001, and European application no. EP 01610061.2 filed on Jun. 8, 2001, the contents of which are fully incorporated herein by reference.

### FIELD OF THE INVENTION

The present invention relates to a mutant DNA sequence encoding peroxisome proliferator-activated receptor-γ coactivator-1 (PGC-1), a method of detecting a mutation in the gene encoding perox isome proliferator-activated receptor-γ coactivator-1, as well as a diagnostic composition and a test kit for use in the method.

### BACKGROUND OF THE INVENTION

Type 2 diabetes, also known as non-insulin dependent diabetes mellitus (NIDDM), is one of the most common of all metabolic disorders and poses a major health problem worldwide. Type 2 diabetes results from defects in both insulin secretion and insulin action, but the exact underlying mechanism(s) causing the disease are not known. An elevation of hepatic glucose production contributes significantly to causing fasting hyperglycemia, whereas decreased insulin-mediated glucose uptake by muscle and fat is a major contributor to postprandial hyperglycemia. Moreover, the metabolic fate of glucose taken up by muscle is not normal in people with type 2 diabetes. For example muscle glycogen synthase activity and glycogen synthesis have been shown to be impaired in type 2 diabetes. The available treatments do not allow for a complete normalisation of the metabolic state and some of them are associated with side effects. The metabolic derangements created by hyperglycemia, together with the strong association between type 2 diabetes, obesity, hypertension, and hyperlipidemia, lead to an extensive list of long-term complications, including a high rate of cardiovascular death due to accelerated atherosclerosis, as well as typical complications of diabetes such as retinopathy, nephropathy, and neuropathy.

There is extensive circumstantial evidence from family investigations including studies in twins and from studies of hybrid populations descended from high- and low-risk ancestral populations in favour of genetic determinants for the common late onset form of type 2 diabetes. It is also likely that type 2 diabetes in many cases is polygenic and it is suggested that subsets of patients display changes in various diabetes susceptibility genes thereby adding to the heterogeneity of type 2 diabetes.

As the symptoms of type 2 diabetes usually occur up to years after the onset of the disease and as type 2 diabetes is often first diagnosed when the long-term complications appear, there is a strong need for methods which enable an earlier diagnosis of type 2 diabetes. One such method could involve the detection of such genetic determinants associated with susceptibility for developing type 2 diabetes.

### SUMMARY OF THE INVENTION

According to the present invention, it has now been found that variability in the PGC-1 (GenBank Accession Number AF106698) gene (SEQ ID NO: 1, the polypeptide encoding part of SEQ ID NO: 1 is situated from nucleotide 121 to nucleotide 2517, including start and stop codons) confers susceptibility to type 2 diabetes and that a widespread missense polymorphism of this gene is reproducibly associated with type 2 diabetes. It is at present assumed that one or more of the mutations may be involved in or associated with the etiology of type 2 diabetes, and their presence may therefore be diagnostic for type 2 diabetes and possibly also other disorders associated with type 2 diabetes, like obesity, hyperlipidemia and hypertension. Without wishing to be bound by any theory, the mutation in PGC-1 associated with type 2 diabetes may be indicative of abnormalities significant for the development of type 2 diabetes or other disorders associated with type 2 diabetes. The mutation may for instance give rise to the substitution of an amino acid in PGC-1 that may cause changes in the tertiary structure of PGC-1. Such changes may interfere with the normal interaction between PGC-1 and the molecules with which it interacts. Mutations may also interfere with the post-translational processing of PGC-1 often resulting in a PGC-1 with an aberrant function. Mutations may also interfere with the transcription or translation of the gene, or with the stability of the PGC-1 transcript. Mutations may also cause defects in splicing of the gene. Alternatively, the mutation may be associated with (i.e. genetically linked with) the mutation or mutations, which causes the disease.

The variability of the gene may be used as a diagnostic tool to identify subjects who are at an increased risk of developing type 2 diabetes. The variant may also identify subjects with variable response to drugs which act via the peroxisome proliferator-activated receptor-γ in other words the variant might be useful for tailoring of antidiabetic medication. The variant may also point to a new gene which could be of importance for development of new drugs.

Accordingly, the present invention encompasses an isolated polynucleotide molecule comprising a nucleotide sequence encoding PGC-1, said nucleotide sequence containing a mutation associated with type 2 diabetes of at least one nucleotide, or comprising a fragment of the nucleotide sequence including said mutation.

The present invention also encompasses a recombinant vector, especially an expression vector, comprising a polynucleotide according to the present invention.

The present invention also encompasses a cell line or a transgenic non-human mammal containing a polynucleotide or a recombinant vector according to the present invention.

The present invention also encompasses a method of detecting the presence of a mutation in the gene encoding PGC-1, the method comprising obtaining a biological sample from a subject and analysing the sample for a mutation associated with type 2 diabetes of at least one nucleotide in the PGC-1 sequence.

The present invention also encompasses a diagnostic composition for determining predisposition to type 2 diabetes, or conditions often associated with type 2 diabetes, in a subject, the composition comprising a polynucleotide according to the present invention.

The present invention also encompasses a test kit for detecting the presence of a mutation associated with type 2 diabetes in the gene encoding PGC-1, the kit comprising a first polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected and optionally a second polynucleotide comprising a nucleotide sequence corresponding to at least part of the wild-type gene encoding PGC-1 and/or optionally a restriction endonuclease, which cleaves DNA at the site of the mutation.

The present invention also encompasses a test kit for detecting the presence of a mutation associated with type 2 diabetes in the gene encoding PGC-1 comprising means for amplifying DNA, and a labelled polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected.

The present invention also encompasses an isolated polypeptide obtainable by expression of a DNA construct comprising a polynucleotide according to the present invention, where said mutation gives rise to an amino acid substitution in PGC-1. The present invention also encompasses an isolated polypeptide, which is a variant of PGC-1 carrying an amino acid substitution associated with type 2 diabetes and which variant is selected from the group consisting of (a) a polypeptide having an amino acid sequence which is substantially homologous to residues 1 to 798 of SEQ ID NO: 2; (b) a polypeptide which is encoded by a polynucleotide comprising a nucleic acid sequence which hybridizes under low stringency conditions with (i) nucleotides 121 to 2514 of SEQ ID NO: 1 or (ii) a subsequence of (i) of at least 100 nucleotides, (c) a variant of a polypeptide comprising an amino acid sequence of SEQ ID NO: 2 comprising a substitution, deletion, and/or insertion of one or more amino acids; (d) an allelic variant of (a) or (b); and (e) a fragment of (a), (b), (c) or (d).

Further embodiments will become apparent from the following detailed description.

### FIGURES

FIG. 1 shows a schematic presentation of identified PGC-1 variants and approximate positions of identified variants relative to known functional domains. LXXLL, recognition site (LXXLL motif); PKAP, protein kinase A phosphorylation consensus site; SRD, serine and arginine rich domain; RRM, RNA recognition motif (Esterbauer et al (1999), Genomics 62, 98-102).

### DEFINITIONS

“Corresponding to”, when used in reference to a nucleotide or amino acid sequence, indicate the position in the second sequence that aligns with the reference position when two sequences are optimally aligned.

The term “isolated”, when applied to a polynucleotide, denotes that the polynucleotide is removed from its natural genetic milieu. Such isolated molecules are those that are separated from the natural environment and include cDNA and genom ic clones. When applied to a protein, the term “isolated” indicates that the polypeptide is found in a condition other than its native environment, such as apart from blood and animal tissue. It may also indicate that the polypetide is chemically synthesized. The isolated polypeptide may be substantially free of other polypeptides, particularly other proteins of animal or plant origin. The polypeptide may be for instance at least about 20% pure, or at least about 40% pure, or at least about 60% pure, or at least about 80% pure, or at least about 90% pure, or at least about 95% pure or at least about 99% pure, as determined for instance by SDS-PAGE.

A “polynucleotide” is a single- or doublestranded polymer of nucleotides such as deoxyribonucleotide or ribonucleotide bases linked together by phosphodiester (5′-3′) bonds and read from the 5′ to the 3′ end. Polynucleotides include RNA and DNA, and may be isolated from natural sources (including genetically engineered organisms), synthesized in vitro, or prepared form a combination of natural and synthetic molecules. It will be recognized by those skilled in the art that the two strands of a double-stranded polynucleotide may differ slightly in length and that the ends thereof may be staggered as a result of enzymatic cleavage; thus all nucleotides within a double-stranded molecule may not be paired. As the skilled person will recognize, this definition of a polynucleotide does also comprise what is known as an oligonucleotide, that is a polynucleotide containing a small number of nucleotides, such as for instance 9, 12, 15, 18, 21, 24, 27, 30 or 33 nucleotides. The polynucleotides of present invention also encompass DNA analogues, such as PNA and LNA.

A “DNA construct” is a polynucleotide as defined above as a single- or doublestranded polymer of deoxyribonucleotide bases.

A “polypeptide” is a linear polymer of amino acids held together by peptide linkages. The polypeptide has a directional sense with an amino and a carboxy terminal end. A polypeptide may be isolated from natural sources (including genetically engineered organisms), synthesized in vitro, or prepared form a combination of natural and synthetic molecules. As the skilled person will recognize, this definition of a polypeptide also comprises what is known as a peptide.

“Operably connected”, when referring to DNA segments, indicates that the segments are arranged so that they function in concert for their intended purposes, e.g. transcription initiates in the promoter and proceeds through the coding segment to the terminator.

### DETAILED DESCRIPTION OF THE INVENTION

The present invention provides an isolated polynucleotide molecule comprising a nucleotide sequence encoding PGC-1, said nucleotide sequence containing a mutation associated with type 2 diabetes of at least one nucleotide, or comprising a fragment of the nucleotide sequence including said mutation.

A polynucleotide molecule comprising a nucleotide sequence encoding PTP-1B also encompasses a polynucleotide which comprises a nucleotide sequence which is substantially homologous to the nucleotide sequence covering nucleotides 91 to 1395 of SEQ ID NO: 1. The term “substantially homologous” is used herein to denote polynucleotides having a sequence identity to the sequence covering nucleotides 91 to 1395 shown in SEQ ID NO: 1 of at least about 65%, or at least about 70%, or at least about 80%, or at least about 90%, or at least about 95%, or at least about 97% while still encoding a polypeptide having an amino acid sequence substantially homologous to residues 1 to 435 of SEQ ID NO: 2. How to determine sequence identity of polynucleotide molecules is described further below.

SEQ ID NO: 1 is the wild-type DNA sequence coding for PGC-1 (GenBank Accession Number AF106698) and SEQ ID NO: 2 is the wild-type amino acid sequence of PGC-1. Those skilled in the art will recognize that the DNA sequence in SEQ ID NO: 1 also provides the RNA sequence encoding SEQ ID NO: 2 by substituting U for T. Those skilled in the art will also readily recognise that, in view of the degeneracy of the genetic code, considerable sequence variation is possible among the polynucleotide molecules according to the present invention. Furthermore, the polynucleotide molecules according to the present invention may contain other sequence variations corresponding to amino acid substitutions in SEQ ID NO: 2 as long as this does not interfere with the utility of the polynucleotides according to the purpose of the present invention. Such sequence variations could for instance correspond to a genetic variation, such as in the form of an allelic variant, within a specific population, a member of which is being diagnosed for susceptibility for developing type 2 diabetes or other disorders associated with type 2 diabetes, like obesity, hyperlipidemia and hypertension, but could also be related to other amino acid substitutions of interest. Those skilled in the art will also recognize that the polynucleotides according to the present invention or a fragment of a polynucleotide according to the present invention may also contain more than one mutation associated with type 2 diabetes or other disorders associated with type 2 diabetes or indeed other mutations of interest.

The length of the polynucleotides according to the present invention may vary widely depending on the intended use. For use as a polynucleotide probe for hybridisation purposes, the polynucleotide may be as short as for instance 17 nucleotides or even shorter. For expression in a cell line or a transgenic non-human mammal as defined above, the polynucleotide according to the present invention will typically comprise the full-length DNA sequence encoding PGC-1. For instance for use in PCR reactions a polynucleotide according to the present invention may comprise additional nucleotides in the N-terminal such as nucleotides forming a restriction endonuclease site for subsequent digestion and cleaving.

The polynucleotide of the present invention comprising the mutation in the nucleotide sequence encoding PGC-1 may suitably be of genom ic DNA or cDNA origin, for instance obtained by preparing a genomic or cDNA library and screening for DNA sequences coding for all or part of the PGC-1 by hybridisation using synthetic oligonucleotide probes in accordance with standard techniques (cf. Sambrook et al., Molecular Cloning: A Laboratory Manual, 2nd Ed., Cold Spring Harbor, Cold Spring Harbor Laboratory Press, 1989). The probes used should be specific for the mutation. Alternatively, the DNA molecule encoding wild-type PGC-1 may be modified by site-directed mutagenesis using synthetic oligonucleotides containing the mutation for homologous recombination in accordance with well-known procedures. The polynucleotides, especially the DNA constructs, according to the present invention, may also be prepared by polymerase chain reaction using specific primers, for instance as described in U.S. Pat. No. 4,683,202, or Saiki et al. (1988), Science 239, 487-491.

The polynucleotides, especially the DNA constructs, of the present invention may also be prepared synthetically by established standard methods, e.g. the phosphoamidite method described by Beaucage and Caruthers (1981), Tetrahedron Letters 22, 1859-1869, or the method described by Matthes et al. (1984), EMBO Journal 3, 801-805. According to the phosphoamidite method, oligonucleotides are synthesized, e.g. in an automatic DNA synthesizer, purified, annealed and ligated. This procedure may preferably be used to prepare fragments of the PGC-1 encoding DNA sequence.

In one embodiment, the present invention provides a polynucleotide according to the present invention comprising a nucleotide sequence as shown in the Sequence Listing as SEQ ID NO: 1 containing a mutation associated with type 2 diabetes of at least one nucleotide or comprising a fragment of the nucleotide sequence shown in the Sequence Listing as SEQ ID NO: 1 including said mutation.

In another embodiment, the present invention provides a polynucleotide according to the present invention, where said mutation gives rise to at least one amino acid substitution in PGC-1.

In another embodiment, the present invention provides a polynucleotide according to the present invention, where said mutation gives rise to a substitution of Gly to an amino acid different from Gly in the position corresponding to position 482 of the amino acid sequence shown in the Sequence Listing as SEQ ID NO: 2.

In another embodiment, the present invention provides a polynucleotide according to the present invention, where said mutation gives rise to a substitution of Gly to Ser in the position corresponding to position 482 of the amino acid sequence shown in the Sequence Listing as SEQ ID NO: 2.

In another embodiment, the present invention provides a polynucleotide according to the present invention, where said mutation corresponds to a mutation of G in position 1564 in SEQ ID NO: 1 to A.

In another embodiment, the present invention provides a polynucleotide according to the present invention where said polynucleotide is a DNA construct.

The present invention provides a recombinant vector, especially an expression vector, comprising a polynucleotide according to the present invention.

The recombinant vector into which a polynucleotide according to the present invention is inserted may be any vector that conveniently may be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmide. Alternatively, the vector may be one which, when introduced into a host cell, is integrated into the host cell genome and replicated together with the chromosome(s) into which it has been integrated (e.g. a viral vector).

In the vector, the mutant DNA sequence encoding PGC-1 may be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the mutant DNA encoding PGC-1 in mammalian cells are the SV40 promoter (Subramani et al. (1981), Mol. Cell Biol. 1, 854-864), the MT-1 (metallothionein gene) promoter (Palmiter et al. (1983), Science 222, 809-814) or the adenovirus 2 major late promoter.

The mutant DNA sequence encoding PGC-1 may also be operably connected to a suitable terminator, such as the human growth hormone terminator (Palmiter et al., ibid.). The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant expression vector may further comprise a DNA sequence enabling the vector to replicate in the host cell in question. An example of such a sequence is the SV40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hygromycin or methotrexate.

The procedures used to ligate the DNA sequences coding for PGC-1, the promoter and the terminator, respectively, and to insert them into suitable vectors containing the information necessary for replication, are well known to persons skilled in the art (cf., for instance, Sambrook et al., ibid.).

The present invention provides a cell line or a transgenic non-human mammal containing a polynucleotide or a recombinant vector according to the present invention.

A cell line into which a polynucleotide or a recombinant vector according to the present invention may be introduced may be any cell in which the polynucleotide can be replicated, such as a prokaryotic cell, for example Eschericia coli or a eukaryotic cell, such as a vertebrate cell, e.g. a Xenopus laevis oocyte or a mammalian cell. The cell line into which a polynucleotide according to the present invention is introduced may also be a cell which is capable of producing PGC-1 and which has the appropriate signal transduction pathways. Such a cell is preferably a eukaryotic cell, such as a vertebrate cell, e.g. a Xenopus laevis oocyte or mammalian cell, in particular a mammalian cell. Examples of suitable mammalian cell lines are the COS (ATCC CRL 1650), BHK (ATCC CRL 1632, ATCC CCL 10), CHL (ATCC CCL39) or CHO (ATCC CCL 61) cell lines.

Methods of transfecting cells, such as prokaryotic cells and eukaryotic cells, such as mammalian cells, are described in e.g. Old RW, Primrose SB, Principles of gene manipulation—an introduction to genetic engineering. Fifth edition. Blackwell Science Ltd. Oxford, 1994.

Expressing DNA sequences introduced in such cells especially eukaryotic cells, and more especially mammalian cells, are described in e.g. Kaufman and Sharp (1982), J. Mol. Biol. 159, 601-621; Southern and Berg (1982), J. Mol. Appl. Genet. 1, 327-341; Loyter et al. (1982), Proc. Natl. Acad. Sci. USA 79, 422-426; Wigler et al. (1978), Cell 14, 725; Corsaro and Pearson (1981), Somatic Cell Genetics 7, 603; Graham and van der Eb (1973), Virology 52, 456; and Neumann et al. (1982), EMBO J. 1, 841-845.

A mutant DNA sequence encoding PGC-1 may then be expressed by culturing cells as described above in a suitable nutrient medium under conditions, which are conducive to the expression of the PGC-1-encoding DNA sequence. The medium used to culture the cells may be any conventional medium suitable for growing such a cell, such as medium suitable for growing mammalian cells, such as a serum-containing or serum-free medium containing appropriate supplements. Suitable media are available from commercial suppliers or may be prepared according to published recipes (e.g. in catalogues of the American Type Culture Collection).

A polynucleotide according to the present invention may also be introduced into a transgenic animal. A transgenic animal is one in whose genome a heterologous DNA sequence has been introduced. In particular, the transgenic animal is a transgenic non-human mammal, mammals being generally provided with appropriate signal transduction pathways. The mammal may conveniently be a rodent such as a rat or mouse. The mutant DNA sequence encoding PGC-1 may be introduced into the transgenic animal by any one of the methods previously described for this purpose. Briefly, the DNA sequence to be introduced may be injected into a fertilised ovum or cell of an embryo, which is subsequently implanted into a female mammal by standard methods, resulting in a transgenic mammal whose germ cells and/or somatic cells contain the mutant DNA sequence. For a more detailed description of a method of producing transgenic mammals, vide B. Hogan et al., Manipulating the Mouse Embryo, Cold Spring Harbor Laboratory, Cold Spring Harbor, N.Y.. The mutant DNA sequence may also be introduced into the animal by transfection of fertilised ova with a retrovirus containing the DNA sequence, cf. R. Jaenisch (1976), Proc. Natl. Acad. Sci. USA 73, 1260-1264. A further method of preparing transgenic animals is described in Gordon and Ruddle ( 1983), Methods Enzymol. 101, 411-432.

The present invention also provides a method of detecting the presence of a mutation in the gene encoding PGC-1, the method comprising obtaining a biological sample from a subject and analysing the sample for a mutation associated with type 2 diabetes of at least one nucleotide in the PGC-1 sequence.

In one embodiment, a method according to the present invention comprises analysing said sample for a mutation associated with type 2 diabetes of at least one nucleotide in the PGC-1 sequence, which mutation gives rise to at least one amino acid substitution in PGC-1.

In another embodiment, a method according to the present invention comprises analysing said sample for a mutation associated with type 2 diabetes of at least one nucleotide in the PGC-1 sequence, which mutation gives rise to a substitution of Gly to an amino acid different from Gly in the position corresponding to position 482 of the amino acid sequence shown in the Sequence Listing as SEQ ID NO: 2.

In another embodiment, a method according to the present invention comprises analysing said sample for a mutation associated with type 2 diabetes of at least one nucleotide in the PGC-1 sequence, which mutation gives rise to a substitution of Gly to Ser in the position corresponding to position 482 of the amino acid sequence shown in the Sequence Listing as SEQ ID NO: 2.

In another embodiment, a method according to the present invention comprises analysing said sample for a mutation associated with type 2 diabetes of at least one nucleotide in the PGC-1 sequence, which mutation corresponds to a mutation of G in position 1564 in SEQ ID NO: 1 to A.

Those skilled in the art will readily recognize that it is within the scope of the present invention to analyse said samples for more than one mutation in PGC-1 associated with type 2 diabetes or other disorders associated with type 2 diabetes or additionally analyse said samples for other mutations in PGC-1 of interest, or indeed for mutations in other genes associated with diabetes or otherwise of interest.

In a further embodiment of a method according to the present invention, a biological sample is obtained from a subject, DNA (in particular genom ic DNA) is isolated from the sample and digested with a restriction endonuclease which cleaves DNA at the site of the mutation, and cleavage of the DNA within the gene encoding PGC-1 at this site is determined. After digestion, the resulting DNA fragments may be subjected to electrophoresis on an agarose gel. DNA from the gel may be visualised, for instance by staining with ethidium bromide. DNA from the gel may also be blotted onto a nitrocellulose filter and hybridised with a labelled probe, such as for instance a radiolabelled probe or a probe labelled as described further below. The probe may conveniently contain a DNA fragment of the PGC-1 gene spanning the mutation (substantially according to the method of E. M. Southern (1975), J. Mol. Biol. 98, 503, e.g. as described by B. J. Conneret al. (1983), Proc. Natl. Acad. Sci. USA 80, 278-282).

Digestion of the DNA may preferably be performed as recommended by the supplier of the enzyme.

In a variant of this embodiment, the DNA isolated from the sample may be amplified prior to digestion with the restriction endonuclease. Amplification may suitably be performed by polymerase chain reaction (PCR) using oligonucleotide primers based on the appropriate sequence of PGC-1 spanning the site(s) of mutation, essentially as described by Saiki et al. (1985), Science 230, 1350-1354. After amplification, the amplified DNA may be digested with the appropriate restriction endonuclease and subjected to agarose gel electrophoresis. The restriction pattern obtained may be analysed, e.g. by staining with ethidium bromide and visualising bands in the gel by means of UV light. As a control, wild-type DNA encoding PGC-1 (i.e. not containing the mutation) may be subjected to the same procedure, and the restriction patterns may be compared.

In one embodiment of a method according to the present invention, a biological sample is obtained from a subject, DNA is isolated from the sample, the DNA is amplified and hybridised to a labelled polynucleotide comprising a nucleotide sequence encoding PGC-1, said nucleotide sequence containing a mutation associated with type 2 diabetes of at least one nucleotide, or comprising a fragment of the nucleotide sequence including said mutation, which mutation corresponds to the mutation the presence of which in the gene encoding PGC-1 is to be detected, and hybridisation of the labelled polynucleotide to the DNA is determined.

In another embodiment of said method, the labelled polynucleotide comprises a nucleotide sequence as shown in the Sequence Listing as SEQ ID NO: 1 containing a mutation associated with type 2 diabetes of at least one nucleotide or comprising a fragment of the nucleotide sequence shown in the Sequence Listing as SEQ ID NO: 1 including said mutation.

In another embodiment of said method, the labelled polynucleotide comprises a nucleotide sequence containing a mutation, which mutation gives rise to an amino acid substitution in PGC-1.

In another embodiment of said method, the labelled polynucleotide comprises a nucleotide sequence containing a mutation, which mutation gives rise to a substitution of Gly to an amino acid different from Gly in the position corresponding to position 482 of the amino acid sequence shown in the Sequence Listing as SEQ ID NO: 2.

In another embodiment of said method, the labelled polynucleotide comprises a nucleotide sequence containing a mutation, which mutation gives rise to a substitution of Gly to Ser in the position corresponding to position 482 of the amino acid sequence shown in the Sequence Listing as SEQ ID NO: 2.

In another embodiment of said method, the labelled polynucleotide comprises a nucleotide sequence containing a mutation corresponding to a mutation of G in position 1564 in SEQ ID NO: 1 to A.

In another embodiment of said method, said labelled polynucleotide is a DNA construct.

In a further embodiment of said method the amplified DNA is hybridised to a second labelled polynucleotide comprising a DNA sequence corresponding to at least part of the wild-type gene encoding PGC-1, and hybridisation of said second labelled polynucleotide to the amplified DNA is determined.

In a further embodiment of said method, the label substance with which the labelled polynucleotide carrying the mutation is labelled is different from the label substance with which the second labelled polynucleotide corresponding to at least part of the wild-type DNA is labelled.

The present invention also encompasses a method according to the present invention for determining predisposition to type 2 diabetes in a subject.

A further embodiment of a method according to the present invention is an adaptation of the method described by U. Landegren et al. (1988), Science 241, 1077-1080, which involves the ligation of adjacent oligonucleotides on a complementary target DNA molecule. Ligation will occur at the junction of the two oligonucleotides if the nucleotides are correctly base paired.

In a further embodiment of a method according to the present invention, the DNA isolated from the sample may be amplified using oligonucleotide primers corresponding to segments of the gene coding for PGC-1. The amplified DNA may then be analysed by hybridisation with a labelled polynucleotide comprising a DNA sequence corresponding to at least part of the gene encoding PGC-1 and containing a mutation of at least one nucleotide, which mutation corresponds to the mutation the presence of which in the gene encoding PGC-1 is to be detected. As a control, the amplified DNA may furthermore be hybridised with a further labelled polynucleotide comprising a DNA sequence corresponding to at least part of the wild-type gene encoding PGC-1. This procedure is, for instance, described by DiLella et al. (1988), Lancet 1, 497-499. Another PCR-based method which may be used in the present invention is the allele-specific PCR method described by R. Saiki et al. (1986), Nature 324, 163-166, or D. Y. Wu et al. (1989), Proc. Natl. Acad. Sci. USA 86, 2757-2760, which uses primers specific for the mutation in the PGC-1 gene.

Other methods of detecting mutations in DNA are reviewed in U. Landegren (1992), GATA 9, 3-8. One of the currently preferred methods of detecting mutations is by single stranded conformation polymorphism (SSCP) analysis substantially as described by Orita et al. (1989), Proc. Natl. Acad. Sci. USA 86, 2766-2770, or Orita et al. (1989), Genomics 5, 874-879 and another is single base extension (also known as microsequencing) substantially as described by Syvänen, A. C. et al. (1992), Genomics 12, 590-5.

The label substance with which a polynucleotide may be labelled may be selected from the group consisting of enzymes, coloured or fluorescent substances, or radioactive isotopes.

Examples of enzymes useful as label substances are peroxidases (such as horseradish peroxidase), phosphatases (such as acid or alkaline phosphatase), β-galactosidase, urease, glucose oxidase, carbonic anhydrase, acetylcholinesterase, glucoamylase, lysozyme, malate dehydrogenase, glucose-6-phosphate dehydrogenase, β-glucosidase, proteases, pyruvate decarboxylase, esterases, luciferase, etc.

Enzymes are not in themselves detectable but must be combined with a substrate to catalyse a reaction the end product of which is detectable. Examples of substrates, which may be employed in the method according to the invention, include hydrogen peroxide/tetramethylbenzidine or chloronaphthole or o-phenylenediamine or 3-(p-hydroxyphenyl) propionic acid or luminol, indoxyl phosphate, p-nitrophenylphosphate, nitrophenyl galactose, 4-methyl umbelliferyl-D-galactopyranoside, or luciferin.

Alternatively, the label substance may comprise coloured or fluorescent substances, including gold particles, coloured or fluorescent latex particles, dye particles, fluorescein, phycoerythrin or phycocyanin.

In one embodiment, the labelled polynucleotide is labelled with a radioactive isotope. Radioactive isotopes, which may be used for the present purpose, may be selected from I-125, I-131, In-111, H-3, P-32, C-14 or S-35. The radioactivity emitted by these isotopes may be measured in a beta- or gamma-counter or by a scintillation camera in a manner known per se.

The present invention provides a diagnostic composition for determining predisposition to type 2 diabetes in a subject, the composition comprising a polynucleotide according to the present invention.

The present invention also provides a diagnostic composition for detecting the presence of a mutation in the gene encoding PGC-1, the composition comprising a polynucleotide according to the present invention.

The present invention also provides a test kit for detecting the presence of a mutation associated with type 2 diabetes in the gene encoding PGC-1, the kit comprising a first polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected and optionally a second polynucleotide comprising a nucleotide sequence corresponding to at least part of the wild-type gene encoding PGC-1 and/or optionally a restriction endonuclease, which cleaves DNA at the site of the mutation.

In one embodiment of said test kit, the first polynucleotide in said test kit is a polynucleotide according to the present invention.

In another embodiment of the present invention, said test kit further comprises means for amplifying DNA.

The present invention also provides a test kit for detecting the presence of a mutation associated with type 2 diabetes in the gene encoding PGC-1, the kit comprising means for amplifying DNA, and a labelled polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected.

In one embodiment of the present invention, the labelled polynucleotide in said test kit comprises a polynucleotide according to the present invention.

In a further embodiment of the present invention, said test kit further comprises a second labelled polynucleotide comprising a nucleotide sequence corresponding to at least part of the wild-type gene encoding PGC-1.

In a further embodiment of the present invention, the label substance with which the labelled polynucleotide in said kit carrying the mutation is labelled is different from the label substance with which the second labelled polynucleotide corresponding to at least part of the wild-type DNA is labelled.

In a further embodiment of the present invention, the second labelled polynucleotide in said test kit is a DNA construct.

In one embodiment, the present invention encompasses a test kit suitable for use in a method according to the present invention.

In one embodiment, the present invention provides a test kit according to the present invention for determining predisposition to type 2 diabetes in a subject.

In one embodiment, the present invention encompasses an isolated polypeptide obtainable by expression of a DNA construct comprising a polynucleotide according to the present invention, where said mutation gives rise to an amino acid substitution in PGC-1. Such a DNA construct may be expressed as part of a recombinant expression vector as described above and as it is generally known in the art. In a further embodiment the amino acid substitution in said isolated polypeptide obtainable by expression of a DNA construct comprising a polynucleotide according to the present invention is a substitution of Gly to an amino acid different from Gly in the position corresponding to position 482 of the amino acid sequence shown in the Sequence Listing as SEQ ID NO: 2. In a further embodiment this amino acid substitution is a substitution of Gly to Ser in the position corresponding to position 482 of the amino acid sequence shown in the Sequence Listing as SEQ ID NO: 2.

The present invention also encompasses an isolated polypeptide, which is a variant of PGC-1 carrying an amino acid substitution associated with type 2 diabetes and which variant is selected from the group consisting of (a) a polypeptide having an amino acid sequence which is substantially homologous to residues 1 to 798 of SEQ ID NO: 2; (b) a polypeptide which is encoded by a polynucleotide comprising a nucleic acid sequence which hybridizes under low stringency conditions with (i) nucleotides 121 to 2514 of SEQ ID NO: 1 or (ii) a subsequence of (i) of at least 100 nucleotides, (c) a variant of a polypeptide comprising an amino acid sequence of SEQ ID NO: 2 comprising a substitution, deletion, and/or insertion of one or more amino acids; (d) an allelic variant of (a) or (b); and (e) a fragment of (a), (b), (c) or (d).

The term “substantially homologous” is used herein to denote polypeptides having a sequence identity to the sequences shown in SEQ ID NO: 2 of at least about 65%, or at least about 70%, or at least about 80%, or at least about 90%, or at least about 95%, or at least about 97% while still having the function of structure of PGC-1. Percent sequence identity is determined by conventional methods. See, for example, Altschul et al., Bull. Math. Bio. 48, 603-616 (1986) and Henikoff and Henikoff, Proc. Natl. Acad. Sci. USA 89, 10915-10919 (1992). Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 1 (amino acids are indicated by the standard one-letter codes). The percent identity is then calculated as:

$$\frac{\text{Total number of identical matches}}{\begin{matrix}\text{[length of the longer sequence plus the number of} \\ \text{gaps introduced into the longer sequence in order} \\ \text{to align the two sequences]}\end{matrix}} \times 100$$

Sequence identity of polynucleotide molecules is determined by similar methods using a ratio as disclosed above.

Substantially homologous polypeptides are characterized as having one or more amino acid substitutions, deletions or additions. These changes are preferably of a minor nature, that is conservative amino acid substitutions (see Table 2) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO J. 4, 1075 (1985); Nilsson et al., Methods Enzymol. 198, 3, 1991), glutathione S transferase (Smith et al., Gene 67, 31 (1988), maltose binding protein (Kellerman et al., Methods Enzymol. 90, 459-463 (1982); Guan et al., Gene 67, 21-30 (1987)), thioredoxin, ubiquitin, cellulose binding protein, T7 polymerase, or other antigenic epitope or binding domain. See, in general Ford et al., Protein Expression and Purification 2, 95-107, 1991, which is incorporated herein by reference. DNAs encoding affinity tags are available from commercial suppliers (e.g., Pharmacia Biotech, Piscataway, N.J.; New England Biolabs, Beverly, Mass.). It is readily apparent to the person skilled in the art that the present invention also encompasses polypeptides according to the present invention, which carry more than one amino acid substitution associated to type 2 diabetes or other disorders associated with type 2 diabetes, like obesity, hyperlipidem ia and hypertension. Similarily, the present invention encompasses polypetides which in addition to one or more amino acid substitutions associated with type 2 diabetes carries other amino acid substitutions of interest such as amino acid substitutions which do significantly affect the folding or activity of the polypeptide.

In addition to the 20 standard amino acids, non-standard amino acids (such as 4-hydroxyproline, 6-N-methyl lysine, 2-aminoisobutyric acid, isovaline and a-methyl serine) may be substituted for PGC-1 amino acid residues. A limited number of non-conservative amino acids, amino acids that are not encoded by the genetic code, and unnatural amino acids may be substituted for PGC-1 amino acid residues. “Unnatural amino acids” have been modified after protein synthesis, and/or have a chemical structure in their side chain(s) different from that of the standard amino acids. Unnatural amino acids can be chemically synthesized, or preferably, are commercially available, and include pipecolic acid, thiazolidine carboxylic acid, dehydroproline, 3- and 4-methylproline, and 3,3-dimethylproline.

For polynucleotides of at least 100 nucleotides in length, low, medium and high stringency conditions are defined as prehybridization and hybridization at 42° C. in 5×SSPE, 0.3% SDS, 200 μg/ml sheared and denatured salmon sperm DNA, and either 25% formamide for low stringency, 35% formamide for medium stringency, or 50% formamide for high stringencies, following standard Southern blotting procedures.

A variant of a polypeptide comprising an amino acid sequence of SEQ ID NO: 2 is a polypeptide which has an amino acid sequence which is substantially similar to the amino acid sequence in SEQ ID NO: 1. Such variants may be the result of modification of a nucleic acid sequence of a polynucleotide according to the present invention which may be desirable for example for increasing the yield of the produced polypeptide or which might otherwise be desirable for handling the polypeptide. The term “substantially similar” to the amino acid sequence refers to amino acid sequences of non-naturally occurring forms of the polypeptide. These polypeptides may differ in some engineered way from PGC-1 as isolated from its native source, e.g., variants that differ in specific activity, thermostability, pH optimum, or the like. The variant sequence may be constructed on the basis of the nucleic acid sequence presented as the polypeptide encoding part of SEQ ID NO: 1, e.g., a subsequence thereof, and/or by introduction of nucleotide substitutions which do not give rise to another amino acid sequence of the polypeptide encoded by the nucleic acid sequence, but which corresponds to the codon usage of the host organism intended for production of the polypeptide, or by introduction of nucleotide substitutions which may give rise to a different amino acid sequence or in other ways. For a general description of nucleotide substitution, see for instance Ford et al., Protein Expression and Purification 2, 95-107 (1991).

An allelic variant denotes any of two or more alternative forms of a gene occupying the same chomosomal locus. Allelic variation arises naturally through mutation, and may result in polymorphism within populations. Gene mutations can be silent (no change in the encoded polypeptide) or may encode polypeptides having altered amino acid sequences. The allelic variant of a polypeptide is a polypeptide encoded by an allelic variant of a gene.

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al. ibid, and Ausubel et al. (eds.), Current Protocols in Molecular Biology, John Wiley and Sons, Inc., New York, 1987, which are incorporated herein by reference.

Polypeptides according to the present invention can be purified using fractionation and/or conventional purification methods and media. Ammonium sulfate precipitation and acid or chaotrope extraction may be used for fractionation of samples. Exemplary purification steps may include differential centrifugation, hydroxyapatite, size exclusion, such as for instance gel filtration, FPLC, ion-exchange chromatography, affinity chromatography, membrane filtration, such as for instance ultrafiltration or diafiltration, or preparative HPLC or any combinations thereof. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Penn.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method is a matter of routine design and is determined in part by the properties of the chosen support. See, for example, Affinity Chromatography: Principles & Methods, Pharmacia LKB Biotechnology, Uppsala, Sweden, 1988.

Protein refolding (and optionally reoxidation) procedures may be advantageously used. It is preferred to purify the protein to at least 80% purity, or to at least 90% purity, or to at least 95%, or to a pharmaceutically pure state, that is at least 99.9% pure with respect to contaminating macromolecules, particularly other proteins, polypeptides and nucleic acids, and free of infectious and pyrogenic agents. Preferably, a purified polypeptide is substantially free of other proteins, particularly other proteins of animal origin.

Polypeptides according to the present invention or fragments thereof may also be prepared through chemical synthesis for instance by use of solid-phase peptide synthesis.

In one embodiment the present invention encompasses an isolated polypeptide, which is a variant of PGC-1 carrying an amino acid substitution associated with type 2 diabetes and which variant is a polypeptide which is encoded by a polynucleotide comprising a nucleic acid sequence which hybridizes under medium stringency conditions with (i) nucleotides 91 to 1395 of SEQ ID NO: 1 or (ii) a subsequence of (i) of at least 100 nucleotides.

In another embodiment the present invention encompasses an isolated polypeptide, which is a variant of PGC-1 carrying an amino acid substitution associated with type 2 diabetes and which variant is a polypeptide which is encoded by a polynucleotide comprising a nucleic acid sequence which hybridizes under high stringency conditions with (i) nucleotides 91 to 1395 of SEQ ID NO: 1 or (ii) a subsequence of (i) of at least 100 nucleotides.

As elucidated in the following example, the PGC-1 gene is associated with type 2 diabetes. Four gene variants of PGC-1 are tested, namely Ser74Leu, IVS2+52C→A, Gly482Ser, and Thr6l2Met were examined in an association study comprising 483 type 2 diabetic patients and 216 glucose tolerant control subjects.

Three of the variants (Ser74Leu, IVS2+52C→A and Thr612Met) showed no significant differences in allele frequencies between diabetic and control subjects. However, the Gly482Ser polymorphism, was more frequent among type 2 diabetic patients (37.0%) compared to glucose tolerant subjects (30.8%) (p=0.032) (Table 4). Testing this variant for association to type 2 diabetes in the second subject samples confirmed the association of the polymorphism to type 2 diabetes. Combining the initial and replication samples demonstrated a 1.34 fold increase in diabetes risk associated with the Ser-allele of Gly482Ser. Although this diabetes susceptibility effect appears to be small it translates into the considerable population attributable risk of 18% due to the high frequency of the risk allele.

PGC-1 Gly482Ser variant is associated with type 2 diabetes in the examined populations and may be the causative polymorphism. Alternatively, the mutation may be a marker associated with another mutation in this or another gene, which other mutation is the one actually involved in disease etiology, for instance in linkage disequilibrium with a not yet identified aetiological variant. Gly482Ser is located in a part of the protein with unknown function and glycine at codon 482 is not conserved between man and mice. Clearly, functional studies are needed to elucidate whether the codon 482 variant itself has biological implications.

### Example 1

Subjects and Methods

Subjects

Mutation analysis was performed in 53 type 2 diabetic patients (30 males, 23 females) recruited from the outpatient clinic at Steno Diabetes Center, Denmark. The age of the patients was 64±9 years, age of diagnosis 57±9 years, body mass index (BMI) 29.7±4.9 kg/m², and HbA_{1C} 8.3±1.7% (mean ± S.D.). More than 70% of the patients fulfilled the WHO criteria for the metabolic syndrome, 31 % of the patients were treated with diet alone, 65% with oral hypoglycaemic agents (OHA), and 4% with insulin alone or in combination with OHA.

The initial association studies were performed in a group of unrelated type 2 diabetic patients recruited from the outpatient clinic at Steno Diabetes Center during 1994-1997 and a group of unrelated glucose tolerant subjects without a known family history of diabetes randomly sampled during 1994-1997 from the Danish Central Population Register and all living in the same area of Copenhagen as the type 2 diabetic patients. In the group of type 2 diabetic patients (n=483, 278 males, 205 females) the age was 61±11 years, age of diagnosis 55±11 years, BMI 29.0±5.3 kg/m², and HbA_{1C} 8.1±1.6%. The patients were treated with diet alone (27%), with OHA (58%), or with insulin in combination with OHA (15%). In the group of glucose tolerant subjects (n=216, 105 males, 111 females) the age was 52±14 years, and BMI 25.3±3.8 kg/m².

The association study used for replication comprised unrelated type 2 diabetic patients recruited from the outpatient clinic at Steno Diabetes Center during 1992-1993 and a population based sample of unrelated glucose tolerant subjects without a known family history of diabetes born in 1936 and examined during 1996-1997 at the Copenhagen County Centre of Preventive Medicine. In the group of type 2 diabetic patients (n=201, 152 males, 49 females) the age was 55±7 years, age of diagnosis 48±8 years, BMI 29.8±4.4 kg m², and HbA_{1C} 8.6±1.7%. The patients were treated with diet alone (29%), with OHA (60%), or with insulin in combination with OHA (11%). In the group of glucose tolerant subjects (n=293, 134 males, 159 females) the age was 60.5±0.4 years and BMI 26.2±3.7 kg/m².

Diabetes was diagnosed according to 1998 WHO criteria (Alberti KG, Zimmet PZ (1998) Definition, diagnosis and classification of diabetes mellitus and its complications. Part 1: diagnosis and classification of diabetes mellitus provisional report of a WHO consultation. Diabet Med 15, 539-553). All glucose tolerant subjects underwent a 75 g oral glucose tolerance test (OGTT). All participants were Danish Caucasians by self-identification. Informed written consent was obtained from all subjects prior to participation. The study was approved by the Ethical Committee of Copenhagen and was in accordance with the principles of the Declaration of Helsinki II.

Biochemical Assays

Blood samples for measurement of serum levels of insulin, total cholesterol, high-density lipoprotein (HDL) cholesterol, triglycerides, and plasma glucose and free fatty acids (FFA) were drawn after a 12-hour overnight fast. Serum triglycerides, total serum cholesterol, serum HDL-cholesterol, serum low-density lipoprotein cholesterol and plasma FFA were analysed using enzymatic colorimetric methods (GPO-PAP and CHOD-PAP, Boehringer Mannheim, Germany and NEFA C, Wako, Germany). The plasma glucose concentration was analysed by a glucose oxidase method (Granutest, Merck, Darmstadt, Germany) and serum specific insulin (excluding des(31,32)- and intact proinsulin) was measured by ELISA (Dako insulin kit K6219, Dako Diagnostics Ltd. Ely, UK). HbA_{1C} was measured by ion-exchange high performance liquid chromatography (non-diabetic reference range: 4.1-6.4%).

Mutation Analysis and Genotypinq

The genetic analyses were performed on genom ic DNA isolated from human leukocytes. The coding region of the PGC-1 gene (EMBL #AF106698) including intron-exon boundaries (in total 3357 bp) was divided into 17 segments (sized 145-273 nucleotides) for SSCP and heteroduplex analysis. In the inventors laboratory this methodology has a sensitivity of more than 95% for detecting a variety of known mutations. The segments also included the 5′ untranslated sequence of 90 bp. Primer sequences are listed in Table PCR amplification was carried out in a volume of 25 μl containing 100 ng genomic DNA, 1×PCR-buffer, 0.2 μM of each primer, 0.2 mM dNTP, 10 mCi/ml α-³²P-dCTP, 0.6 units AmpliTaq Gold polymerase (Perkin Elmer, Calif., USA) and MgCl₂ concentration as shown in Table 3. The cycling program was a denaturation step at 95° C. for 15 min followed by 40 cycles of 94° C. for 30 seconds, annealing at Tₐₙₙₑₐₗ for 30 seconds, and elongation at 72° C. for 60 seconds with a final elongation step at 72° C. for 9 min using a GeneAmp 9600 thermal cycler (Perkin Elmer). The annealing temperatures are listed in Table 3. SSCP was performed at two different experimental settings as reported in Hansen T et al. (1997), Diabetes 46, 494-501 and aberrantly migrating samples were sequenced using fluorescent chemistry (Dye Primer Cycle Sequencing Ready Reaction Kit, Applied Biosystems, California, USA). The Ser74Leu and IVS2+52C→A variants were genotyped by PCR with primers PC2F-PC2RNY followed by digestion with Dral and Apal, respectively. The Gly482Ser variant was amplified with primers PC15F-PC17R and digested with HpaII. The Thr612Met variant was amplified with primers PC8F-PC8R and digested with NlaIII. All restriction enzyme digests were separated on 4% agarose gels.

Statistical Analysis

Fisher's exact test was applied to examine for differences in allele frequencies between diabetic and non-diabetic subjects. A p-value of less than 0.05 was considered significant. All analyses were performed using Statistical Package for Social Science (SPSS) version 10.0. The genotype relative risk (GRR) was estimated by logistic regression from the genotype data using a log-additive model for the risk. Test for additivity gave a likelihood ratio statistic of 0.285 on 1 df (p=0.593). Population attributable risk was calculated in standard fashion as

(P_{He}·(GRR−1)+P_{Ho}·(GRR²−1))/(1+P_{He}·(GRR−1)+P_{Ho}·(GRR²−1)),

where P is the frequency of the risk genotype.

Results

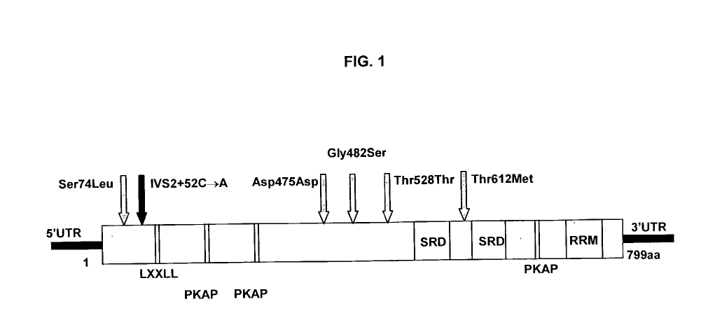

The mutation screening covered the coding region of PGC-1. In the 53 diabetic patients a total of six different variants was identified (FIG. 1): Ser74Leu (identified in 2 out of 53 patients), IVS2+52C→A (19 patients), Asp475Asp (13 patients), Gly482Ser (24 patients), Thr528Thr (37 patients), and Thr6l2Met (3 patients). The three variants, which predicted changes of amino acids and the prevalent intronic variant, IVS2+52C→A, were further examined in an association study comprising 483 type 2 diabetic patients and 216 glucose tolerant control subjects. All variants were in Hardy-Weinberg equilibrium. The allele frequencies of the Ser74Leu, IVS2+52C→A, and Thr6l2Met variants did not differ significantly between diabetic and non-diabetic subjects (Table 4).

The allele frequency of the Gly482Ser variant was higher among type 2 diabetic patients compared to glucose tolerant subjects (37.0% vs. 30.8%, p=0.032) (Table 4). In a replication study the differences in allele frequencies remained significant (38.1% vs. 30.4%, p=0.0135). The combined study yielded an allelic frequency of 37.3% for the type 2 diabetic patients and 30.5% for the glucose tolerant subjects (p=0.0007). The genotype relative risk for diabetes was estimated to 1.34 (95% confidence interval: 1.13-1.59) corresponding to a population attributable risk of 18%. Assuming an effect of the variant of 18% the combined study has a statistical power of approximately 90% for detecting a difference in allele frequency of the Gly482Ser polymorphism.

In the combined group of diabetic subjects, carriers of the Gly482Ser polymorphism did not differ significantly from wild type carriers in clinical or biochemical values including age of diabetes onset, BMI, waist circumference, treatment, degree and prevalence of micro- and macrovascular complications, HbA_{1C} or fasting serum lipids (data not shown). Moreover, in the glucose tolerant subjects there was no evidence of a relation between the codon 482 variant and estimates of BMI, waist circumference, fasting serum triglycerides, serum total and HDL-cholesterol, plasma free fatty acids or plasma glucose, serum insulin and serum C-peptide during an OGTT (data not given).

TABLE 1

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

TABLE 2
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | Phenylalanine |
| | Tryptophan |
| | Tyrosine |
| Small: | Glycine |
| | Alanine |
| | Serine |
| | Threonine |
| | Methionine |

TABLE 3

Primer sequences and PCR conditions for mutational analysis of PGC-1

| | | | | |
|---|---|---|---|---|
| Primer | Sequence, 5′→3′ | Location | Tₐₙₙₑₐₗ | MgCl₂ |

| | | | | | |
|---|---|---|---|---|---|
| PC1F | CTG GGG ACT GTA GTA AGA C | (SEQ ID NO.3) | 5′UTR + | 55° C. | 1 mM |
| | | Exon 1 | | | |
| PC1R | AGG GAA GCG TCA GTT GTG G | (SEQ ID NO.4) | | | |
| PC2F | CCT GTG GTT AAT GGA AGC | (SEQ ID NO.5) | Exon 2 | 50° C. | 2 mM |
| PC2RNY | GCC CAA GCC AAA CTC AAT G | (SEQ ID NO.6) | | | |
| PC3F | CTG CCT CCC AGG GTC AAC | (SEQ ID NO.7) | Exon 3 | 55° C. | 1 mM |
| PC3R | CAA CTC CAA TTC CTG CTA AAC | (SEQ ID NO.8) | | | |
| PC4F | GAT GCA TAA CTT TAG TTG | (SEQ ID NO.9) | Exon 4 | 50° C. | 2 mM |
| PC4R | CTG CTT CAA GCC AAA ATC | (SEQ ID NO.10) | | | |
| PC5F | CTG ATA AGG TTC AGT TCA C | (SEQ ID NO.11) | Exon 5 | 50° C. | 2 mM |
| PC5R | CCT CAC CAA CAG CTC GT | (SEQ ID NO.12) | | | |
| PC6F | CCA ACT TGA CTG TTG TGG AG | (SEQ ID NO.13) | Exon 6 | 55° C. | 2 mM |
| PC6R | ACA AAC TGA AAT GGA GTT GC | (SEQ ID NO.14) | | | |
| PC7F | GGG TTC TAA TAC ATT TGG C | (SEQ ID NO.15) | Exon 7 | 50° C. | 2 mM |
| PC7R | CAC ATA GAC AGT ACA TCT | (SEQ ID NO.16) | | | |
| PC8F | GTT AAG TGG CAG TTG CAA ATG | (SEQ ID NO.17) | Exon 9 | 55° C. | 2 mM |
| PC8R | GGG AGC TAA AGG AAA ATG AC | (SEQ ID NO.18) | | | |
| PC9F | GGT GGT TGA CTT AGT GAT AAA G | (SEQ ID NO.19) | Exon 10 | 55° C. | 3 mM |
| PC9R | CAC AGA AAA AGA AGA AAC CCT AC | (SEQ ID NO.20) | | | |
| PC10F | CCA CTC CAG AAC TCT CTC C | (SEQ ID NO.21) | Exon 11 | 55° C. | 1 mM |
| PC10R | CAA CTC CCA TCC CAG TAA TC | (SEQ ID NO.22) | | | |
| PC11F | GGT TAC AGT CCC ATA TAC T | (SEQ ID NO.23) | Exon 12 | 50° C. | 3 mM |
| PC11R | GAT TCC TCA TTC CAC GTA C | (SEQ ID NO.24) | | | |
| PC12F | GCC ATC AGC AAA GTG TGT | (SEQ ID NO.25) | Exon 13 | 50° C. | 2 mM |
| PC12R | TGA GGT ATT CGC CAT CCC | (SEQ ID NO.26) | | | |
| PC13F | GAA ACA TGT GTC TTC GCA | (SEQ ID NO.27) | Exon 8 | 55° C. | 2 mM |
| PC14R | CGC TTG GTC TTC CTT TCC TCG | (SEQ ID NO.28) | | | |
| PC15F | CAA GTC CTC AGT CCT CAC | (SEQ ID NO.29) | Exon 8 | 50° C. | 2 mM |
| PC15R | CTT GCC TCC AAA GTC TCT C | (SEQ ID NO.30) | | | |
| PC16F | CAG ATT CAG ACC AGT G | (SEQ ID NO.31) | Exon 8 | 45° C. | 1 Mm |
| PC16R | CAT AGG TAG TTT GGA G | (SEQ ID NO.32) | | | |
| PC17F | GGG ACA GTG ATT TCA GTA ATG | (SEQ ID NO.33) | Exon 8 | 55° C. | 1 mM |
| PC17R | GGG GTC TTT GAG AAA ATA AGG | (SEQ ID NO.34) | | | |
| PC18F | GTA GAG ATT CTG TGT CAC | (SEQ ID NO.35) | Exon 8 | 45° C. | 2 mM |
| PC18R | CTT TTG TGT TAT TTA GGG | (SEQ ID NO.36) | | | |

All forward primers were extended with a 21M13 tail for sequencing (TGT AAA ACG ACG GCC AGT) and all reverse primers with an M13 tail (CAG GAA ACA GCT AGT ACC)

TABLE 4
Genotype and allele frequencies of the examined variants in the PGC-1
gene in type 2 diabetic patients and glucose tolerant subjects

| | Type 2 | | Glucose | | |
|---|---|---|---|---|---|
| | diabetic patients | | tolerant subjects | | p |
| | Initial association study | | | | |
| Ser74Leu | | | | | |
| Ser/Ser | 466 | (99) | 197 | (99) | |
| Ser/Leu | 3 | (1) | 1 | (1) | |
| Leu/Leu | 0 | (0) | 0 | (0) | |
| Allele frequency | 0.3 | (0-0.7) | 0.3 | (0-0.7) | 1.0 |
| IVS2 + 52C→A | | | | | |
| C/C | 178 | (37) | 62 | (30) | |
| C/A | 221 | (46) | 102 | (50) | |
| A/A | 79 | (17) | 40 | (20) | |
| Allele frequency | 39.6 | (36.6-42.7) | 44.6 | (39.8-49.4) | 0.09 |
| Thr612Met | | | | | |
| Thr/Thr | 443 | (93) | 183 | (90) | |
| Thr/Met | 31 | (7) | 20 | (10) | |
| Met/Met | 1 | (0) | 0 | (0) | |
| Allele frequency | 3.5 | (2.3-4.6) | 4.9 | (2.8-7.0) | 0.2 |
| Gly482Ser | | | | | |
| Gly/Gly | 186 | (41) | 97 | (49) | |
| Gly/Ser | 200 | (44) | 80 | (40) | |
| Ser/Ser | 68 | (15) | 21 | (11) | |
| Allele frequency | 37.0 | (33.8-40.1) | 30.8 | (26.2-35.3) | 0.032 |
| | Replication study | | | | |
| Gly482Ser | | | | | |
| Gly/Gly | 76 | (38) | 146 | (50) | |
| Gly/Ser | 97 | (48) | 116 | (40) | |
| Ser/Ser | 28 | (14) | 31 | (10) | |
| Allele frequency | 38.1 | (33.3-42.8) | 30.4 | (26.7-34.0) | 0.0135 |
| | Combined study | | | | |
| Gly482Ser | | | | | |
| Gly/Gly | 262 | (40) | 243 | (49) | |
| Gly/Ser | 297 | (45) | 196 | (40) | |
| Ser/Ser | 96 | (15) | 52 | (11) | |
| Allele frequency | 37.3 | (34.7-39.9) | 30.5 | (27.7-33.4) | 0.0007 |

Data are number of subjects with each genotype (% of each group) and allele frequencies of minor allele in % (95% confidence interval). The p-values compare allele frequencies between type 2 diabetic patients and glucose tolerant subjects.

What is claimed is:

1. An isolated polynucleotide molecule selected from the group consisting of: (i) a polynucleotide comprising a nucleotide sequence encoding PGC-1, wherein said nucleotide sequence contains a mutation of at least one nucleotide relative to the wild-type sequence of PGC-1 and wherein said mutation is associated with type 2 diabetes, and (ii) a polynucleotide comprising a fragment of the nucleotide sequence encoding PGC-1, wherein said fragment contains a mutation of at least one nucleotide relative to the wild-type sequence of PGC-1 and wherein said mutation is associated with type 2 diabetes.

2. A polynucleotide according to claim 1, wherein said wild-type sequence encoding PGC-1 is the sequence of SEQ ID NO: 1.

3. A polynucleotide according to claim 1, where said mutation gives rise to at least one amino acid substitution in the PGC-1 polypeptide relative to the amino acid sequence of wild-type PGC-1.

4. A polynucleotide according to claim 3, wherein said mutation gives rise to a substitution of Gly to an amino acid different from Gly in the position corresponding to position 482 of the amino acid sequence of SEQ ID NO: 2.

5. A polynucleotide according to claim 3, wherein said mutation gives rise to a substitution of Gly to Ser in the position corresponding to position 482 of the amino acid sequence of SEQ ID NO: 2.

6. A polynucleotide according to claim 1, wherein said mutation corresponds to a mutation of G in position 1564 in SEQ ID NO: 1 to A.

7. A polynucleotide according to claim 1, wherein said polynucleotide is a DNA construct.

8. A recombinant vector comprising a polynucleotide according to claim 1.

9. A cell line or a transgenic non-human mammal comprising a polynucleotide according to claim 1.

10. A cell line according to claim 9, wherein the cell line is a mammalian cell line.

11. A method of detecting the presence of a mutation in a gene encoding PGC-1, the method comprising (i) obtaining a biological sample from a subject and (ii) analysing the sample for a mutation of at least one nucleotide in the PGC-1 sequence, wherein said mutation is associated with type 2 diabetes.

12. A method according to claim 11, wherein the mutation gives rise to at least one amino acid substitution in PGC-1.

13. A method according to claim 12, wherein the mutation gives rise to a substitution of Gly to an amino acid different from Gly in the position corresponding to position 482 of the amino acid sequence of SEQ ID NO: 2.

14. A method according to claim 13, wherein the mutation gives rise to a substitution of Gly to Ser in the position corresponding to position 482 of the amino acid sequence of SEQ ID NO: 2.

15. A method according to claim 11, wherein the mutation corresponds to a mutation of G in position 1564 in SEQ ID NO: 1 to A.

16. A method according to claim 11, wherein said analysing comprises: (i) isolating DNA from the sample, (ii) amplifying the DNA and (iii) hybridising the amplified DNA to a labelled polynucleotide comprising a nucleotide sequence encoding PGC-1 or a fragment thereof, wherein said polynucleotide comprises a mutation associated with type 2 diabetes of at least one nucleotide, and (iv) determining hybridisation of the labelled polynucleotide to the amplified DNA.

17. A method according to claim 16, wherein the labelled polynucleotide comprises (i) a nucleotide sequence as shown in the Sequence Listing as SEQ ID NO: 1 containing a mutation associated with type 2 diabetes of at least one nucleotide or (ii) a fragment of the nucleotide sequence shown in the Sequence Listing as SEQ ID NO: 1 including said mutation.

18. A method according to claim 16, wherein the mutation gives rise to at least one amino acid substitution in PGC-1.

19. A method according to claim 16, wherein the mutation gives rise to a substitution of Gly to an amino acid different from Gly in the position corresponding to position 482 of the amino acid sequence of SEQ ID NO: 2.

20. A method according to claim 16, wherein the mutation gives rise to a substitution of Gly to Ser in the position corresponding to position 482 of the amino acid sequence of SEQ ID NO: 2.

21. A method according to 16, wherein the mutation corresponds to a mutation of G in position 1564 in SEQ ID NO: 1 to A.

22. A method according to claim 16, wherein said labelled polynucleotide is a DNA construct.

23. A method according to claim 16, further comprising hybridising the amplified DNA to a second labelled polynucleotide, wherein said second polynucleotide comprises a DNA sequence corresponding to at least part of the wild-type gene encoding PGC-1, and determining hybridisation of said second labelled polynucleotide to the amplified DNA.

24. A method according to claim 23, wherein the label substance with which the labelled polynucleotide carrying the mutation is labelled is different from the label substance with which the second labelled polynucleotide corresponding to at least part of the wild-type DNA is labelled.

25. A method according to claim 11, wherein said method identifies predisposition to type 2 diabetes in a subject.

26. A diagnostic composition for determining predisposition to type 2 diabetes in a subject, the composition comprising a polynucleotide according to claim 1.

27. A diagnostic composition for detecting the presence of a mutation in the gene encoding PGC-1, the composition comprising a polynucleotide according to claim 1.

28. A test kit for detecting the presence of a mutation associated with type 2 diabetes in the gene encoding PGC-1, the kit comprising
(a) a first polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected and, optionally
(b) a second polynucleotide comprising a nucleotide sequence corresponding to at least part of the wild-type gene encoding PGC-1, and/or optionally
(c) a restriction endonuclease, which cleaves PGC-1-encoding DNA at the site of the mutation.

29. A test kit according to claim 28, wherein the first polynucleotide is a polynucleotide.

30. A test kit according to claim 28, further comprising a means for amplifying DNA.

31. A test kit for detecting the presence of a mutation associated with type 2 diabetes in the gene encoding PGC-1, the kit comprising
(a) means for amplifying DNA, and
(b) a labelled polynucleotide comprising a nucleotide sequence corresponding to at least part of the gene encoding PGC-1 and containing a mutation of at least one nucleotide, which mutation corresponds to the mutation the presence of which in the gene encoding PGC-1 is to be detected.

32. A test kit according to claim 31, wherein the labelled polynucleotide comprises a polynucleotide.

33. A test kit according to claim 31, which comprises a second labelled polynucleotide comprising a nucleotide sequence corresponding to at least part of the wild-type gene encoding PGC-1.

34. A test kit according to claim 33, wherein the label substance with which the labelled polynucleotide carrying the mutation is labelled is different from the label substance with which the second labelled polynucleotide corresponding to at least part of the wild-type DNA is labelled.

35. A test kit according to claim 33, wherein the second labelled polynucleotide is a DNA construct.

36. A test kit suitable for use in a method according to any of claims 11 to 25.

37. A test kit according to any of claims 28 to 36 for determining predisposition to type 2 diabetes in a subject.

38. An isolated polypeptide encoded by a polynucleotide according to claim 3.

39. An isolated polypeptide selected from the group consisting of
(a) a polypeptide having an amino acid sequence which is substantially homologous to residues 1 to 798 of SEQ ID NO: 2;
(b) a polypeptide which is encoded by a polynucleotide comprising a nucleic acid sequence which hybridizes under low stringency conditions with (i) nucleotides 121 to 2514 of SEQ ID NO: 1 or (ii) a subsequence of (i) of at least 100 nucleotides,
(c) a variant of a polypeptide comprising an amino acid sequence of SEQ ID NO: 2 comprising a substitution, deletion, and/or insertion of one or more amino acids;
(d) an allelic variant of (a) or (b); and
(e) a fragment of (a), (b), (c) or (d).
wherein said isolated polypeptide is a variant of PGC-1 carrying an amino acid substitution associated with type 2 diabetes


SEQUENCE LISTING (36 sequences)

SEQ ID NO: 1 - DNA, 6303 bp, Homo Sapiens
  polyA_signal (5164)...(5169): gene = PPARGC1
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacggaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacacaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacacttaaaacctagaacttcaaaatgttcgtattctattcaaaaggaaaaatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattattttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcaaaataattcttccagtgcaacaaaatcaattttttgctaaacgactccaagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatct

SEQ ID NO: 2 - protein, 798 aa, Homo Sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 3 - DNA, 19 bp, Artificial Sequence
  Synthetic
ctggggactgtagtaagac

SEQ ID NO: 4 - DNA, 19 bp, Artificial Sequence
  Synthetic
agggaagcgtcagttgtgg

SEQ ID NO: 5 - DNA, 18 bp, Artificial Sequence
  Synthetic
cctgtggttaatggaagc

SEQ ID NO: 6 - DNA, 19 bp, Artificial Sequence
  Synthetic
gcccaagccaaactcaatg

SEQ ID NO: 7 - DNA, 18 bp, Artificial Sequence
  Synthetic
ctgcctcccagggtcaac

SEQ ID NO: 8 - DNA, 21 bp, Artificial Sequence
  Synthetic
caactccaattcctgctaaac

SEQ ID NO: 9 - DNA, 18 bp, Artificial Sequence
  Synthetic
gatgcataactttacttg

SEQ ID NO: 10 - DNA, 18 bp, Artificial Sequence
  Synthetic
ctgcttcaagccaaaatc

SEQ ID NO: 11 - DNA, 19 bp, Artificial Sequence
  Synthetic
ctgataaggttcagttcac

SEQ ID NO: 12 - DNA, 17 bp, Artificial Sequence
  Synthetic
cctcaccaacagctcgt

SEQ ID NO: 13 - DNA, 20 bp, Artificial Sequence
  Synthetic
ccaacttgactgttgtggag

SEQ ID NO: 14 - DNA, 20 bp, Artificial Sequence
  Synthetic
acaaactgaaatggagttgc

SEQ ID NO: 15 - DNA, 19 bp, Artificial Sequence
  Synthetic
gggttctaatacatttggc

SEQ ID NO: 16 - DNA, 18 bp, Artificial Sequence
  Synthetic
cacatagacagtacatct

SEQ ID NO: 17 - DNA, 21 bp, Artificial Sequence
  Synthetic
gttaagtggcagttgcaaatg

SEQ ID NO: 18 - DNA, 20 bp, Artificial Sequence
  Synthetic
gggagctaaaggaaaatgac

SEQ ID NO: 19 - DNA, 22 bp, Artificial Sequence
  Synthetic
ggtggttgacttagtgataaag

SEQ ID NO: 20 - DNA, 23 bp, Artificial Sequence
  Synthetic
cacagaaaaagaagaaaccctac

SEQ ID NO: 21 - DNA, 19 bp, Artificial Sequence
  Synthetic
ccactccagaactctctcc

SEQ ID NO: 22 - DNA, 20 bp, Artificial Sequence
  Synthetic
caactcccatcccagtaatc

SEQ ID NO: 23 - DNA, 19 bp, Artificial Sequence
  Synthetic
ggttacagtcccatatact

SEQ ID NO: 24 - DNA, 19 bp, Artificial Sequence
  Synthetic
gattcctcattccacgtac

SEQ ID NO: 25 - DNA, 18 bp, Artificial Sequence
  Synthetic
gccatcagcaaagtgtgt

SEQ ID NO: 26 - DNA, 18 bp, Artificial Sequence
  Synthetic
tgaggtattcgccatccc

SEQ ID NO: 27 - DNA, 18 bp, Artificial Sequence
  Synthetic
gaaacatgtgtcttcgca

SEQ ID NO: 28 - DNA, 21 bp, Artificial Sequence
  Synthetic
cgcttggtcttcctttcctcg

SEQ ID NO: 29 - DNA, 18 bp, Artificial Sequence
  Synthetic
caagtcctcagtcctcac

SEQ ID NO: 30 - DNA, 19 bp, Artificial Sequence
  Synthetic
cttgcctccaaagtctctc

SEQ ID NO: 31 - DNA, 16 bp, Artificial Sequence
  Synthetic
cagattcagaccagtg

SEQ ID NO: 32 - DNA, 16 bp, Artificial Sequence
  Synthetic
cataggtagtttggag

SEQ ID NO: 33 - DNA, 21 bp, Artificial Sequence
  Synthetic
gggacagtgatttcagtaatg

SEQ ID NO: 34 - DNA, 21 bp, Artificial Sequence
  Synthetic
ggggtctttgagaaaataagg

SEQ ID NO: 35 - DNA, 18 bp, Artificial Sequence
  Synthetic
gtagagattctgtgtcac

SEQ ID NO: 36 - DNA, 18 bp, Artificial Sequence
  Synthetic
cttttgtgttatttaggg